(19) United States
(12) Patent Application Publication
(10) Pub. No.: US 2020/0327889 A1
(43) Pub. Date: Oct. 15, 2020

(54) NURSE OPERATION ASSISTANCE TERMINAL, NURSE OPERATION ASSISTANCE SYSTEM, NURSE OPERATION ASSISTANCE METHOD, AND NURSE OPERATION ASSISTANCE PROGRAM RECORDING MEDIUM

(71) Applicant: NEC CORPORATION, Tokyo (JP)

(72) Inventors: Yuan LUO, Tokyo (JP); Masahiro KUBO, Tokyo (JP); Masahiro HAYASHITANI, Tokyo (JP); Yuji OHNO, Tokyo (JP); Yutaka UNO, Tokyo (JP); Shigemi KITAHARA, Tokyo (JP)

(73) Assignee: NEC CORPORATION, Tokyo (JP)

(21) Appl. No.: 16/756,336

(22) PCT Filed: Oct. 5, 2018
(86) PCT No.: PCT/JP2018/037386

§ 371 Date: Apr. 15, 2020

(30) Foreign Application Priority Data
Oct. 16, 2017 (JP) 2017-200218

(51) Int. Cl. G10L 15/22; G10L 15/08; G10L 15/26; G16H 40/20; G06N 20/00

(52) U.S. Cl. CPC G10L 15/22; G10L 15/08; G10L 15/265; G16H 40/20; G10L 2015/088; G10L 2015/223; G10L 2015/225; G10L 2015/227; G06N 20/00

(57) ABSTRACT

The present invention addresses the problem of improving a nursing operation. The nurse operation assistance terminal is provided with: a presentation unit which presents, to a nurse at the site of treatment and in sequence, necessary items of treatment to be performed with respect to a patient; and an input unit into which a response speech the nurse has uttered in response to the presented necessary items is input, and which outputs a response speech signal indicating the response speech as an input value. A recording device is provided with: a nursing necessity degree table for recoding a nursing necessity degree; a speech identification means which identifies the input value of the response speech signal as an identification result comprising text data; and a recording unit which automatically records the identification result in a corresponding predetermined field in the nursing necessary degree table.

### TECHNICAL FIELD

The present invention relates to a nurse operation assistance terminal, a nurse operation assistance system, a nurse operation assistance method, and a nurse operation assistance program recording medium.

### BACKGROUND ART

In a hospital or a nursing home, as part of operations of a nurse or a careworker (which will simply be called a “nurse” hereinafter), an entry of a nursing necessity degree for a patient or a care receiver (which will simply be called a “patient” hereinafter) is carried out every day. Under existing circumstances, the nurse enters the nursing necessity degree based on judgment of each individual nurse, using a user terminal while watching a display screen for the nursing necessity degree. However, depending on a difference in judgment among individual nurses, there are variations in score of the nursing necessity degree. In addition, the entry of the nursing necessity degree is carried out by each individual nurse using the user terminal after treatment for the patient is finished. That is, the entry of the nursing necessity degree is not recorded simultaneously with the treatment. Therefore, there are a lot of cases where mistaken memory of the nurse (especially, a young nurse) causes incomplete entry or erroneous entry of the nursing necessity degree.

In view of the above, in order to resolve the incomplete entry or the erroneous entry caused by the difference in judgment among the individual nurses and the mistaken memory, various technical ideas have been proposed.

For example, Patent Literature 1 discloses a technical idea of properly supporting a work relevant to a nursing necessity degree. In Patent Literature 1, with respect to a specific date, a nursing necessity degree for a patient is registered in a nursing necessity degree database (DB) in association with duty time zones of at least two types or more among day shift, evening shift, and midnight shift. A screen generation unit acquires the highest nursing necessity degree in the duty time zones of two or more types and produces the acquired nursing necessity degree as a specific-date's nursing necessity degree for the patient via an input/output processing unit. Thus, support relevant to the nursing necessity degree is properly supported.

Patent Literature 2 discloses a technical idea of enabling reduction in labor of inputting information necessary for calculating a nursing necessity degree. In Patent Literature 2, a nurse call information output unit produces nurse call information based on warning information related to abnormality detection by medical equipment that is produced in a pseudo manner by a warning information output unit or out-of-use information of the medical equipment that is produced by an out-of-use information output unit. A nursing necessity degree check data generation unit generates, based on the nurse call information, nursing necessity degree check data to be used for calculating the nursing necessity degree for a patient. With values supplied to items related with use of the medical equipment for the patient, the nursing necessity degree check data generation unit generates the nursing necessity degree check data.

Although not a system for supporting a nurse operation, Patent Literature 3 discloses an “automatic inquiry system” for guiding a patient to give assumed answers to inquiries when he or she gives unexpected answers. The automatic inquiry system disclosed in Patent Literature 3 has a structure in which a management server, a diagnostician computer, and a patient terminal device are communicably connected via a network. The patient terminal device includes a speaker, a microphone, a speech output unit, a speech recognition unit, a question read unit, an answer discrimination unit, and an answer preservation unit. The question read unit successively reads question information stored in a question information storage unit to make the speech output unit utter question sentences via the speaker. The answer discrimination unit discriminates whether or not answer information, which is supplied by the patient via the microphone immediately after outputting utterance of each of the question sentences and which is converted by the speech recognition unit into textual information, is the assumed answer being a selection from options assumed in response to the immediately preceding question sentence. When it is discriminated that the answer information is the assumed answer, the answer preservation unit preserves the answer information in an inquiry result storage unit in association with the question sentence. When an answer is supplied via the microphone, the speech recognition unit converts an input speech into a text format and supplies it to the answer discrimination unit. The answer discrimination unit carries out discrimination by judging whether or not a specific keyword is included in such answer sentence.

Likewise, although not a system for supporting a nurse operation, Patent Literature 4 discloses an “equipment control device” which can contribute to equipment control by accurately judging a state of mind of a worker that changes as time elapses. In Patent Literature 4, reported information is supplied to a speech recognition unit using a speech supplied via a microphone. When the reported information is supplied by means of the speech, utterance of words which are preliminarily determined is basically carried out from a point of view of accuracy. After a physiological index and the reported information are supplied at the same time instant, one of judgment rules that corresponds to a current combination is referred to. When there is a corresponding rule, the state of mind is judged. In addition, in Patent Literature 4, a necessary rule is acquired by learning. For example, when teaching of failure is supplied, the learning is carried out by: deducing which judgment was a problem backward to the past; decreasing a confidence weight of the judgment rule adopted at that time by a fixed amount or rate; and relatively increasing confidence weights of others which are not realized.

### CITATION LIST

### Patent Literatures

PL 1: JP 2016-153966 A
PL 2: JP 2009-075872 A
PL 3: JP 2010-094243 A
PL 4: JP 2008-000497 A

### SUMMARY OF INVENTION

### Technical Problem

However, Patent Literatures 1 to 4 described above have problems which will be described in the following.

Patent Literature 1 merely discloses the technical idea for confirming, by a head nurse or the like, whether or not the nursing necessity degree is properly recorded by a nurse. Accordingly, in Patent Literature 1, the entry of the nursing necessity degree is carried out by the individual nurse using a user terminal after treatment for the patient is finished. As a result, in the technical idea disclosed in Patent Literature 1, incomplete entry can be prevented but it is impossible to resolve a mistaken memory and to shorten (reduce) an entry operation using the user terminal.

The technical idea disclosed in Patent Literature 2 merely covers a part of the items (items related to use of the equipment) in the nursing necessity degree to be entered. That is, in Patent Literature 2, the items related to use of the equipment can be recorded in a linked manner but it is impossible to record the nursing necessity degree in the linked manner with respect to measures in non-use of the equipment.

Patent Literature 3 proposes the automatic inquiry system for preserving the answer information in association with the question sentence when the question information is successively read, the question sentence is outputted via the speaker as utterance and, immediately after the output of utterance, it is discriminated that the answer information obtained by inputting by the patient via the microphone is the assumed answer. Accordingly, Patent Literature 3 merely discloses the automatic inquiry system for obtaining an inquiry result from the patient and does not support the nurse operation of recording, by the nurse, the nursing necessity degree to be entered. In particular, the “inquiry” in Patent Literature 3 is carried out quite regardless of a “treatment behavior” performed by the nurse on the patient and therefore is a matter (concept) which is quite different from the “nursing necessity degree” associated with the “treatment behavior”.

Patent Literature 4 merely discloses the technical idea of judging the state of mind of a user using the physiological index acquired from the user and the reported information reported by the user. In particular, the “reported information” in Patent Literature 4 is based on the utterance of the words which are preliminarily determined. Accordingly, the “reported information” in Patent Literature 4 is quite different from, for example, a “usual conversation” exchanged between the nurse and the patient. Furthermore, the “judgment rule” in Patent Literature 4 is used for judging the state of mind of the user. Accordingly, the “judgment rule” in Patent Literature 4 is not intended to recognize (extract) useful information, for example, from the above-mentioned usual conversation (e.g. a keyword for specifying the “nursing necessity degree”).

It is an object of the present invention to resolve the above-mentioned problems and to provide a nurse operation assistance terminal, a nurse operation assistance system, a nurse operation assistance method, and a nurse operation assistance program recording medium, which are capable of improving a nursing operation.

### Solution to Problem

According to a first aspect of the present invention, a nurse operation assistance terminal comprises a presentation unit configured to sequentially present, to a nurse at a treatment site, necessary items with which a patient is to be treated; and an input unit configured to input response speeches uttered by the nurse in response to the presented necessary items and to output input values.

A nurse operation assistance system according to the present invention comprises the above-mentioned nurse operation assistance terminal; and a recording device configured to receive the input values from the nurse operation assistance terminal, wherein the recording device comprises a nursing necessity degree table configured to record the nursing necessity degree; a speech identification means configured to identify the input values being response speech signals as identified results being text data; and a recording unit configured to automatically record the identified results in corresponding predetermined fields on the nursing necessity degree table.

According to a second aspect of the present invention, the nurse operation assistance terminal is characterized in that the input unit comprises a speech identification means configured to identify the inputted response speeches and to output the input values as identified results being text data.

A nurse operation assistance system according to the present invention comprises the above-mentioned nurse operation assistance terminal; and a recording device configured to receive the identified results from the nurse operation assistance terminal, wherein the recording device comprises a nursing necessity degree table configured to record the nursing necessity degree; and a recording unit configured to automatically record the identified results in corresponding predetermined fields on the nursing necessity degree table.

According to a third aspect of the present invention, a nurse operation assistance terminal comprises a collection unit configured to collect, as a collected speech, utterance generated by a nurse to a patient and/or conversation exchanged between the nurse and the patient in conjunction with a treatment behavior performed on the patient by the nurse at a treatment site; and an output means configured to output a collected speech signal indicative of the collected speech.

A nurse operation assistance system according to the present invention comprises the above-mentioned nurse operation assistance terminal; and a registration system configured to receive the collected speech signal from the nurse operation assistance terminal, wherein the registration system comprises a treatment behavior acquisition device configured to acquire treatment behavior information from the collected speech signal; a judgment device configured to judge a nursing necessity degree based on the treatment behavior information to produce a judged value; and a recording device configured to record the judged value.

A nurse operation assistance method according to the present invention comprises acquiring, by a treatment behavior acquisition device, treatment behavior information from a collected speech signal received from a nurse operation assistance terminal; contrasting, by an information contract means, the treatment behavior information with predetermined necessary items to produce a plurality of keywords required to specify a nursing necessity degree; judging, by a judgment means, the nursing necessity degree, based on a judgment rule and using a combination of the plurality of keywords, to produce a judged value; and automatically recording, by a recording unit, the judged value in a corresponding predetermined field on a nursing necessity degree table.

A nurse operation assistance program recording medium according to the present invention records a nurse operation assistance program which causes a computer to execute a treatment behavior acquisition step of acquiring treatment behavior information from a collected speech signal received from a nurse operation assistance terminal; an information contrast step of contrasting the treatment behavior information with predetermined necessary items to produce a plurality of keywords required to specify a nursing necessity degree; a judgment step of judging the nursing necessity degree, based on a judgment rule and using a combination of the plurality of keywords, to produce a judged value; and a recording step of automatically recording the judged value in a corresponding predetermined field on a nursing necessity degree table.

According to a third aspect of the present invention, a nurse operation assistance terminal comprises a collection unit configured to collect utterance generated by a nurse to a patient and/or a conversation exchanged between the nurse and the patient in conjunction with a treatment behavior performed on the patient by the nurse and to produce a collected speech signal indicative of a collected speech; and a treatment behavior acquisition unit configured to acquire treatment behavior information from the collected speech signal.

A nurse operation assistance system according to the present invention comprises the above-mentioned nurse operation assistance terminal; and a registration system configured to receive the treatment behavior information from the nurse operation assistance terminal, wherein the registration system comprises a judgment device configured to judge a nursing necessity degree based on the treatment behavior information to produce a judged value; and a recording device configured to record the judged value

A nurse operation assistance method according to the present invention comprises contrasting, by an information contract means, treatment behavior information received from a nurse operation assistance terminal with predetermined necessary items to produce a plurality of keywords required to specify a nursing necessity degree; judging, by a judgment means, the nursing necessity degree, based on a judgment rule and using a combination of the plurality of keywords, to produce a judged value; and automatically recording, by a recording unit, the judged value in a corresponding predetermined field on a nursing necessity degree table.

A nurse operation assistance program recording medium according to the present invention records a nurse operation assistance program which causes a computer to execute an information contract step of contrasting treatment behavior information received from a nurse operation assistance terminal with predetermined necessary items to produce a plurality of keywords required to specify a nursing necessity degree; a judgment step of judging the nursing necessity degree, based on a judgment rule and using a combination of the plurality of keywords, to produce a judged value; and a recording step of automatically recording the judged value in a corresponding predetermined field on a nursing necessity degree table.

### Advantageous Effect of the Invention

According to the present invention, it is possible to improve a nursing operation.

### BRIEF DESCRIPTION OF THE DRAWINGS

FIG. 1 is a block diagram for illustrating a configuration of a nurse operation assistance system according to a first example embodiment of the present invention;

FIG. 2 is a view for illustrating an example of a nursing necessity table used in the nurse operation assistance system illustrated in FIG. 1;

FIG. 3 is a flow chart for illustrating an example of a flow of operation of the nurse operation assistance system according to the first example embodiment of the present invention;

FIG. 4 is a block diagram for illustrating a configuration of a nurse operation assistance system according to a modification (first modification) of the first example embodiment of the present invention;

FIG. 5 is a block diagram for illustrating a configuration of a nurse operation assistance system according to a second example embodiment of the present invention;

FIG. 6 is a flow chart for illustrating an example of a flow of operation of the nurse operation assistance system according to the second example embodiment of the present invention;

FIG. 7 is a block diagram for illustrating a configuration of a nurse operation assistance system according to a modification (second modification) of the second example embodiment of the present invention;

FIG. 8 is a block diagram for illustrating a configuration of a nurse operation assistance system according to a third example embodiment of the present invention;

FIG. 9 is a view for illustrating an example of a nursing necessity table used in the nurse operation assistance system illustrated in FIG. 8;

FIG. 10 is a view for illustrating an example of training data required for training a learning unit used in the nurse operation assistance system illustrated in FIG. 8;

FIG. 11 is a flow chart for illustrating an example of a flow of operation of the nurse operation assistance system according to the third example embodiment of the present invention;

FIG. 12 is a block diagram for illustrating a configuration of a nurse operation assistance system according to a modification (third modification) of the third example embodiment of the present invention;

FIG. 13 is a block diagram for illustrating a configuration of a nurse operation assistance system according to a fourth example embodiment of the present invention;

FIG. 14 is a flow chart for illustrating an example of a flow of operation of the nurse operation assistance system according to the fourth example embodiment of the present invention;

FIG. 15 is a block diagram for illustrating a configuration of a nurse operation assistance system according to a modification (fourth modification) of the fourth example embodiment of the present invention; and

FIG. 16 is a block diagram for illustrating an example of hardware configuration of a registration system of the nurse operation assistance system according to the third or the fourth example embodiment (third or fourth modification) of the present invention.

### DESCRIPTION OF EMBODIMENTS

Now, example embodiments of the present invention will be described in detail with reference to the drawings. Note that, in respective figures, the same or the corresponding parts are assigned with the same symbols and description of overlapping portions will be omitted as appropriate.

### First Example Embodiment

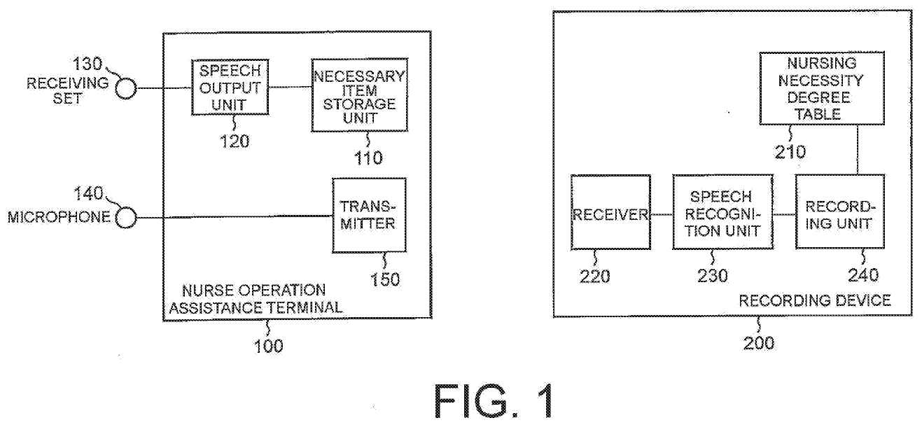

FIG. 1 is a block diagram for illustrating a configuration of a nurse operation assistance system according to a first example embodiment of the present invention. As illustrated in FIG. 1, the nurse operation assistance system comprises a nurse operation assistance terminal 100 and a recording device 200.

In the first example embodiment, the following case is supposed. In the illustrated nurse operation assistance system, a case where a specific nurse treats a specific patient in a predetermined time zone is supposed. In this case, it is necessary to enter a nursing necessity degree in a nursing necessity degree table for the specific patient in question. However, it is to be noted that, as is well known, the patient ID (identifier) is assigned to each patient and, accordingly, there are nursing necessity degree tables which are different for every patient ID in reality. In addition, treatment to be performed on the specific patient by the specific nurse in the above-mentioned time zone is preliminarily determined.

In addition, the first example embodiment exemplifies a case of entering the nursing necessity degree of the specific patient by real-time processing. However, the present invention is not limited to the real-time processing, and is applicable to batch processing as will later be described.

The nurse operation assistance terminal 100 is a terminal which can automatically input the nursing necessity degree of the specific patient at a treatment site. Accordingly, the nurse operation assistance terminal 100 illustrated in the figure is for exclusive use of the specific patient and is assigned with a patient ID for the specific patient. In other words, in the example being illustrated, the nurse operation assistance terminal 100 is provided for every patient. The nurse operation assistance terminal 100 comprises a necessary item storage unit 110, a speech output unit 120, a receiving set 130, a microphone 140, and a transmitter 150. The receiving set 130 may be an earphone or a headphone.

As described above, the nurse operation assistance terminal 100 is the nurse operation assistance terminal for the specific patient. Accordingly, in the above-mentioned predetermined time zone, the specific nurse in charge of the specific patient carries the nurse operation assistance terminal 100 for the specific patient and goes from a nurse center or the like to a sickroom or the like in which the specific patient is present. In other words, a plurality of nurse operation assistance terminals which are different from one another for a plurality of patients are prepared in the nurse center or the like. In addition, since the specific nurse is required to perform treatment on the specific patient, it is desirable to use an earphone with a microphone or a headphone with a microphone as a combination of the receiving set 130 and the microphone 140. Alternatively, the nurse operation assistance terminal 100 may be kept close to the specific patient.

In the necessary item storage unit 110, speech data indicating a plurality of treatments to be performed on the specific patient in the predetermined time zone are preliminarily stored in accordance with an order in which the respective treatments are to be performed. For example, such speech data may be read out of a predetermined database and only necessary information required during the above-mentioned predetermined time zone is preliminarily stored in the necessary item storage unit 110. Instead, in a case where the nurse operation assistance terminal 100 is kept close to the specific patient, the following may be performed. First, from a control center which is not shown in the figure, necessary items for the specific patient are read out of the predetermined database in the predetermined time zone and transmitted to the nurse operation assistance terminal 100. The nurse operation assistance terminal 100 may comprise a receiver (not shown) for receiving the transmitted necessary items and store the received necessary items in the necessary item storage unit 110. Alternatively, in the nurse assistance terminal 100, the necessary items received by the receiver may be directly supplied to the speech output unit 120 without using the necessary item storage unit 110.

The speech output unit 120 sends the speech data read out of the necessary item storage unit 110 to the receiving set 130. The receiving set 130 converts the speech data into a question speech. Accordingly, a combination of the speech output unit 120 and the receiving set 130 serves as a speech notification means for notifying the specific nurse of the necessary items with the question speech. In addition, a combination of the necessary item storage unit 110, the speech output unit 120, and the receiving set 130 acts as a presentation unit for sequentially presenting, to the specific nurse, the necessary items to be performed on the specific patient.

In response to the question speech generated from the receiving set 130, the specific nurse utters a response speech corresponding thereto towards the microphone 140. For example, it is assumed that “is there a wound treatment?” as the question speech flows from the receiving set 130. In this event, the specific nurse replies, for example, “yes” with a speech towards the microphone 140. The microphone 140 converts the response speech into an input value of a response speech signal which is an electric signal. Accordingly, the microphone 140 serves as an input unit configured to input the response speech uttered by the specific nurse in response to the presented necessary items, and to output the input value (response speech signal).

The transmitter 150 receives the input value (response speech signal) sent from the microphone 140. The transmitter 150 adds the patient ID to the input value, modulates a carrier using the signal with the addition, and transmits a modulated wave as a transmitted wave to the recording device 200 wirelessly.

In the example being illustrated, the nurse operation assistance terminal 100 and the recording device 20 are wirelessly connected to each other using Bluetooth (registered trademark), WiFi, or the like. However, a wireless connection method is not limited thereto.

The recording device 200 comprises a nursing necessity degree table 210, a receiver 220, a speech recognition unit 230, and a recording unit 240.

As described above, the recording device 200 is a recording device for the specific patient. Accordingly, the nursing necessity degree table 210 is a nursing necessity degree table for the above-mentioned specific patient. Therefore, the first example embodiment comprises the recording device 200 for every patient.

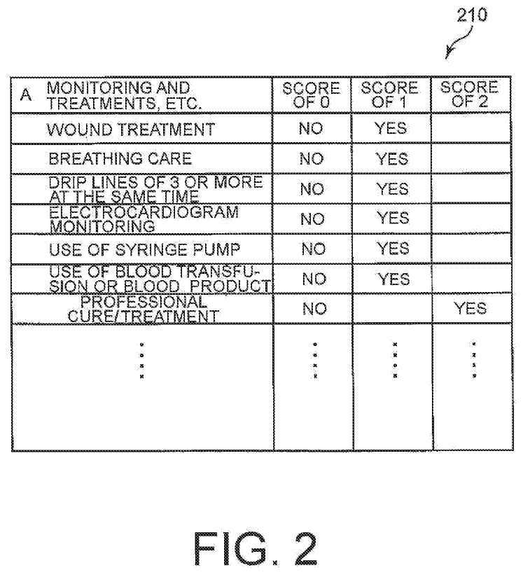

FIG. 2 is a view for illustrating a specific example of the nursing necessity degree table 210. The illustrated nursing necessity degree table 210 includes a plurality of items related to A: monitoring and treatments, etc. Each of the items is given a score of zero if “no” or a score of one or two if “yes”.

Turning back to FIG. 1, the receiver 220 receives, as a received wave, the transmitted wave transmitted from the transmitter 150 of the nurse operation assistance terminal 100. The receiver 220 modulates the received wave to reproduce the above-mentioned input value (response speech signal). The reproduced input value is supplied to the speech recognition unit 230. The speech recognition unit 230 recognizes the input value to produce a recognized result as an identified result being text data. Accordingly, the speech recognition unit 230 serves as a speech identification means configured to identify the input value being the response speech signal as the identified result being the text data.

The recording unit 240 receives the identified result from the speech recognition unit 230. The recording unit 240 automatically records the identified result in a corresponding predetermined field on the nursing necessity degree table 210. Specifically, as described above, it is assumed that, in the nurse operation assistance terminal 100, the specific nurse replies “yes” as the response speech in response to the question speech “is there a wound treatment?” In this event, the speech recognition unit 230 recognizes (identifies) “yes” for an item of “wound treatment”. As a result, the recording unit 240 records “yes” in the field of “wound treatment” in the nursing necessity degree table 210.

Now, effects of the first example embodiment will be described. As described above, the nurse operation assistance terminal 100 according to the first example embodiment can automatically input, at the treatment site, the necessary items required as the nursing necessity degree of the specific patient. As a result, it is possible to reliably eliminate a difference in judgment among individuals and the mistaken memory of the nurse. Furthermore, the recording device 200 automatically enters the items inputted from the nurse operation assistance terminal 100 in the nursing necessity degree table 210. Accordingly, it is possible to reduce the operation of entering the nursing necessity degree by the nurse using the user terminal that is carried out in the related art.

[Operation of Nurse Operation Assistance System]

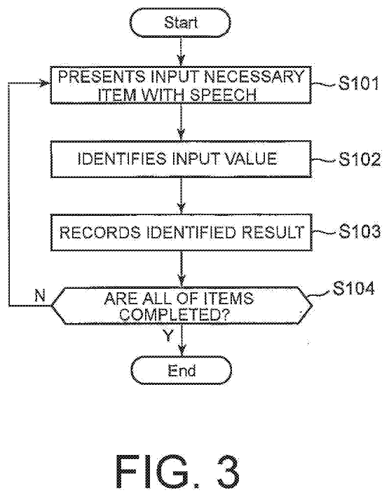

FIG. 3 is a flow chart for illustrating a flow of operation of the nurse operation assistance system illustrated in FIG. 1. Now, the flow of the operation of the nurse operation assistance system will be described with reference to FIGS. 1 and 3.

First, the speech output unit 120 of the nurse operation assistance terminal 100 reads speech data of one necessary item out of the necessary item storage unit 110 to present it with the question speech from the receiving set 130 (Step S101).

In response to the question speech, the response speech uttered by the specific nurse is transmitted through the microphone 140 and the transmitter 150 to the recording device 200 as the transmitted wave indicative of the input value. The receiver 220 of the recording device 200 receives the transmitted wave as the received wave and demodulates it to reproduce the response speech signal (input value).

Next, the speech recognition unit 230 of the recording device 200 identifies the input value as the identified result (Step S102).

Next, the recording unit 240 records the identified result in the nursing necessity degree table 210 (Step S103).

The nurse operation assistance system repeats the above-mentioned processing (Steps S101 to S103) until all of necessary items are read out of the necessary item storage unit 110 (Step S104).

Although the nurse operation assistance terminals 100 are prepared for the respective patients in the above-mentioned first example embodiment, the present invention is not limited thereto. For example, one nurse operation assistance terminal 100 may be prepared for a plurality of patients if positions and/or names of the patients can be identified (specified). Furthermore, when the names of the patients can be identified, it is unnecessary to preliminarily determine treatments to be performed on the patients with respect to the predetermined time zone.

[First Modification]

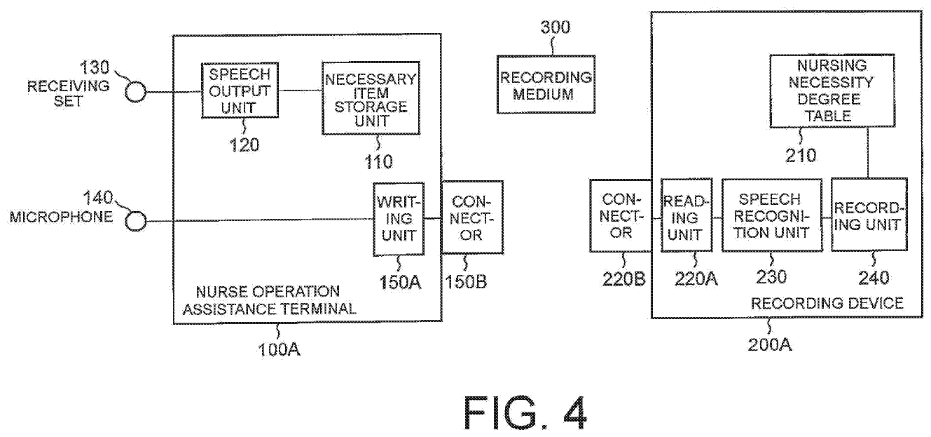

Referring now to FIG. 4, description will proceed to a nurse operation assistance system according to a modification (first modification) of the first example embodiment. Although the case of the real-time processing is described in the above-mentioned first example embodiment by way of example, batch processing which will be described hereafter may be applied.

In a case of the batch processing, a recording medium 300, such as a memory stick or a USB (Universal Serial Bus) memory, is used. Such a recording medium is prepared for every patient. Accordingly, the illustrated recording medium 300 is a recording medium for the specific patient and is assigned with the patient ID for the specific patient.

In addition, in the nurse operation assistance system according to the first modification, the nurse operation assistance terminal 100 and the recording device 200 illustrated in FIG. 1 are changed into a nurse operation assistance terminal 100A and a recording device 200A, respectively, as follows. Specifically, the nurse operation assistance terminal 100A uses a writing unit 150A and a terminal connector 150B in lieu of the transmitter 150. The writing unit 150A is inserted and connected between the terminal connector 150B and the microphone 140. On the other hand, the recording device 200A uses a reading unit 220A and a recording connector 220B in lieu of the receiver 220. The reading unit 220A is inserted and connected between the recording connector 220B and the speech recognition unit 230.

First, the above-mentioned recording medium 300 for the specific patient is inserted into the terminal connector 150B of the nurse operation assistance terminal 100A at a time instant before the above-mentioned predetermined time zone. In this state, when the predetermined time zone is reached, the input value (response speech signal) inputted from the microphone 140 is sent to the writing unit 150A. The writing unit 150A adds the necessary item, a writing time instant, and the patient ID to the input value (response speech signal) and writes the added information as recorded information in the recording medium 300 for the specific patient via the terminal connector 150B.

The recording medium 300 for the specific patient, with the above-mentioned recorded information written therein, is inserted into the recording connector 220B of the recording device 200A at a suitable time instant after lapse of the above-mentioned time zone. In the recording device 200A, the reading unit 220A reads, out of the recording medium 300 for the specific patient, the recorded information written therein via the recording connector 220B. The read-out recorded information is supplied to the speech recognition unit 230. Since the read-out recorded information includes the patient ID, the writing time instant, and the necessary item as mentioned above, the speech recognition unit 230 can identify the input value as the identified result for every necessary item.

As described above, the batch processing is carried out. The first modification also has effects similar to those of the above-mentioned first example embodiment. That is, in the first modification also, it is possible to reliably eliminate a difference in judgment among individuals and the mistaken memory of the nurse and to reduce the entering operation.

Although the recording medium 300 is provided outside in the above-mentioned first modification, the recording medium 300 may be installed inside the nurse operation assistance terminal 100A. In this event, it is necessary for the terminal connector 150B of the nurse operation assistance terminal 100A to be directly connected to the recording connector 220B of the recording device 200A at the suitable time instant after lapse of the above-mentioned time zone. Accordingly, in order to achieve this, one and the other of the terminal connector 150B and the recording connector 220B are modified into a male type and a female type, respectively, so as to be fitted with each other.

Furthermore, the batch processing may be achieved without using the recording medium 300. In order to achieve this, the nurse operation assistance system of a wireless communication type illustrated in FIG. 1 may be modified as follows. First, in the nurse operation assistance system 100, the transmitter 150 may add, to the input value, not only the patient ID but also the necessary item and the writing time instant, modulate the carrier using a signal with those additions, and transmit the modulated wave as the transmitted wave to the recording device 200 wirelessly. On the other hand, the recording device 200 may comprise a buffer memory for temporarily storing the response speech signal (input value) transmitted from the nurse operation assistance terminal 100 without directly supplying it to the speech recognition unit 230. Then, at the suitable time instant after lapse of the above-mentioned time zone, the response speech signals (input values) are successively supplied from the buffer memory to the speech recognition unit 230.

### Second Example Embodiment

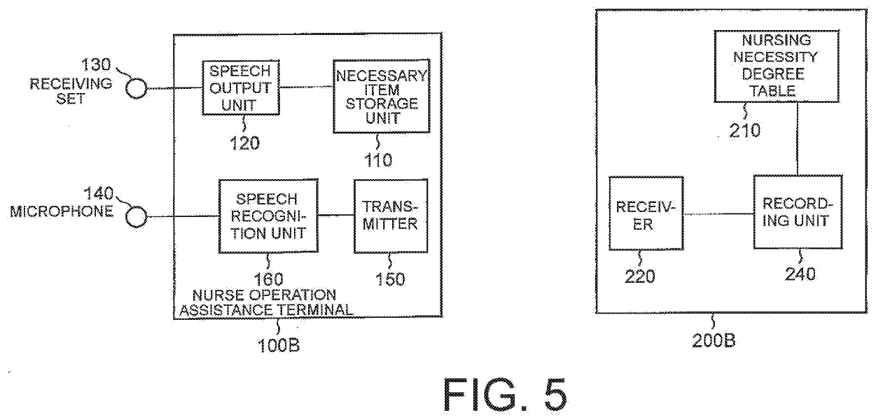

FIG. 5 is a block diagram for illustrating a configuration of a nurse operation assistance system according to a second example embodiment of the present invention. As illustrated in FIG. 5, the nurse operation assistance system comprises a nurse operation assistance terminal 100B and a recording device 200B.

In the second example embodiment also, a case similar to that in the above-mentioned first embodiment is supposed. That is, in the illustrated nurse operation assistance system, a case where a specific nurse treats a specific patient in a predetermined time zone is supposed.

In addition, like in the first example embodiment, the second example embodiment also exemplifies a case of entering the nursing necessity degree of the specific patient by real-time processing. However, the present invention is not limited to the real-time processing, and is applicable to batch processing also.

A difference between the above-mentioned first example embodiment and the second example embodiment exists in a signal which is transmitted and received between the nurse operation assistance terminal and the recording device. In detail, in the above-mentioned first example embodiment, the response speech signals inputted from the microphone are transmitted and received as the input values. On the other hand, in the second example embodiment, the identified results are transmitted and received as the input values, as will later be described.

The nurse operation assistance terminal 100B is similar in structure and operation to the nurse operation assistance terminal 100 illustrated in FIG. 1 except that a speech recognition unit 160 is further provided. That is, the nurse operation assistance terminal 100B comprises the necessary item storage unit 110, the speech output unit 120, the receiving set 130, the microphone 140, the transmitter 150, and the speech recognition unit 160.

The speech recognition unit 160 is inserted between the microphone 140 and the transmitter 150. The speech recognition unit 160 recognizes the response speech signal supplied from the microphone 140 to produce a recognized result as an identified result being text data. Accordingly, the speech recognition unit 160 serves as a speech identification means configured to identify the input response speech to produce the input value as the identified result being the text data.

The identified result is supplied to the transmitter 150. Accordingly, the transmitter 150 adds the patient ID to the identified result, modulates a carrier using a signal with the addition, and transmits the modulated wave to the recording device 200B as the transmitted wave wirelessly.

In the example being illustrated also, the nurse operation assistance terminal 100B and the recording device 200B are wirelessly connected to each other using Bluetooth, WiFi, or the like. However, a wireless connection method is not limited to thereto.

The recording device 200B is similar in structure and operation to the recording device 200 illustrated in FIG. 1 except that the speech recognition unit 230 is deleted. That is, the recording device 200B comprises the nursing necessity degree table 210, the receiver 220, and the recording unit 240. The receiver 220 and the recording unit 240 are directly connected to each other.

The receiver 220 receives, as a received wave, the transmitted wave transmitted from the transmitter 150 of the nurse operation assistance terminal 100B. The receiver 220 demodulates the received wave to reproduce the above-mentioned identified result. The reproduced identified result is supplied to the recording unit 240. The recording unit 240 automatically records the identified result in a corresponding predetermined field on the nursing necessity degree table 210.

In addition, the nursing necessity table 210 stores contents illustrated in FIG. 2 in the manner similar to the above-mentioned first example embodiment.

Now, effects of the second example embodiment will be described. As described above, the nurse operation assistance terminal 100B according to the second example embodiment can automatically input, at the treatment site, the necessary items required as the nursing necessity degree of the specific patient. As a result, it is possible to reliably eliminate a difference in judgment among individuals and the mistaken memory of the nurse. Furthermore, the recording device 200B automatically enters the items inputted from the nurse operation assistance terminal 100B in the nursing necessity degree table 210. Accordingly, it is possible to reduce the operation of entering the nursing necessity degree by the nurse using the user terminal that is carried out in the related art.

[Operation of Nurse Operation Assistance System]

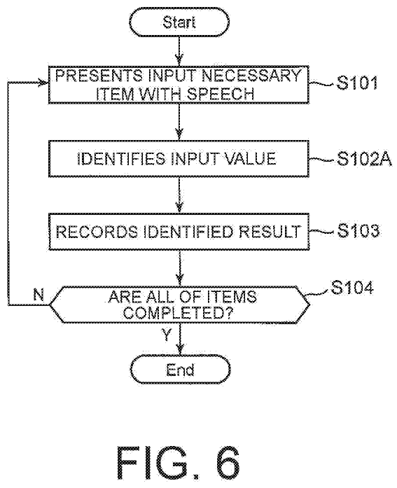

FIG. 6 is a flow chart for illustrating a flow of operation of the nurse operation assistance system illustrated in FIG. 5. Now, the flow of the operation of the nurse operation assistance system will be described with reference to FIGS. 5 and 6.

First, the speech output unit 120 of the nurse operation assistance terminal 100B reads speech data of one necessary item out of the necessary item storage unit 110 to present it with the question speech from the receiving set 130 (Step S101).

The response speech (input value) uttered by the specific nurse in response to the question speech is converted by the microphone 140 into the response speech signal which is transmitted to the speech recognition unit 160. The speech recognition unit 160 identifies the input value (response speech signal) as the identified result (Step S102A).

The identified result is transmitted through the transmitter 150 to the recording device 200B as the transmitted wave. The receiver 220 of the recording device 200B receives the transmitted wave as the received wave and demodulates it to reproduce the identified result.

The reproduced identified result is supplied to the recording unit 240. The recording unit 240 records the identified result in the nursing necessity degree table 210 (Step S103).

The nurse operation assistance system repeats the above-mentioned processing (Steps S101, S102A, and S103) until all of necessary items are read out of the necessary item storage unit 110 (Step S104).

Although the nurse operation assistance terminals 100B are prepared for the respective patients in the above-mentioned second example embodiment also, the present invention is not limited thereto. As described above, for example, one nurse operation assistance terminal 100B may be prepared for a plurality of patients if positions and/or names of the patients can be identified (specified). Similarly, when the names of the patient can be identified, it is unnecessary to preliminarily determine treatments to be performed on the patients with respect to the predetermined time zone.

### Second Modification

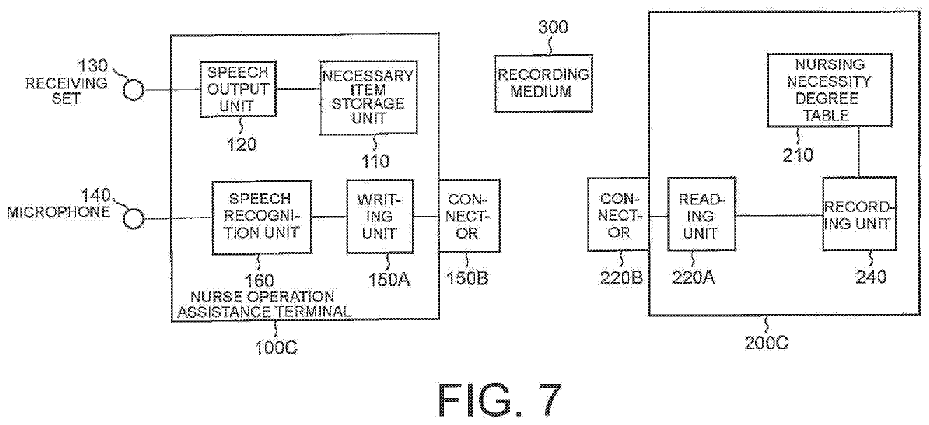

Referring now to FIG. 7, description will proceed to a nurse operation assistance system according to a modification (second modification) of the second example embodiment. Although the case of the real-time processing is described in the above-mentioned second example embodiment by way of example, batch processing which will be described hereafter may be applied.

In a case of the batch processing, a recording medium 300, such as a memory stick or a USB memory, is used as described above. Such a recording medium is prepared for every patient. Accordingly, the illustrated recording medium 300 is a recording medium for the specific patient and is assigned with the patient ID for the specific patient.

In addition, in the nurse operation assistance system illustrated in FIG. 7, the nurse operation assistance terminal 100B and the recording device 200B illustrated in FIG. 5 are changed into a nurse operation assistance terminal 100C and a recording device 200C, respectively. Specifically, the nurse operation assistance terminal 100C uses the writing unit 150A and the terminal connector 150A in lieu of the transmitter 150. The writing unit 150A is inserted and connected between the terminal connector 150B and the speech recognition unit 160. On the other hand, the recording device 200B uses the reading unit 220A and the recording connector 220B in lieu of the receiver 220. The reading unit 220B is inserted and connected between the recording connector 220B and the recording unit 240.

First, the above-mentioned recording medium 300 for the specific patient is inserted into the terminal connector 150B of the nurse operation assistance terminal 100C at a time instant before the above-mentioned predetermined time zone. In this state, the input value (identified result) sent from the speech recognition unit 160 is sent to the writing unit 150A in the predetermined time zone. The writing unit 150A adds the necessary item, a writing time instant, and the patient ID to the input value (identified result) and writes the added information as recorded information in the recording medium 300 for the specific patient via the terminal connector 150B.

The recording medium 300 for the specific patient, with the above-mentioned recorded information written therein, is inserted into the recording connector 220B of the recording device 200C at a suitable time instant after lapse of the above-mentioned time zone. In the recording device 200C, the reading unit 220A reads, out of the recording medium 300 for the specific patient, the recorded information written therein via the recording connector 220B. The read-out recorded information is supplied to the recording unit 240. Since the read-out recorded information includes the patient ID, the writing time instant, and the necessary item as mentioned above, the recording unit 240 can receive the identified result for every necessary item.

As described above, the batch processing is carried out. The second modification also has effects similar to those of the above-mentioned second example embodiment. That is, in the second modification also, it is possible to reliably eliminate a difference in judgment among individuals and the mistaken memory of the nurse and to reduce the entering operation.

Although the recording medium 300 is provided outside in the above-mentioned second modification, the recording medium 300 may be installed inside the nurse operation assistance terminal 100C. In this event, it is necessary for the terminal connector 150B of the nurse operation assistance terminal 100C to be directly connected to the recording connector 220B of the recording device 200C at the suitable time instant after lapse of the above-mentioned time zone. Accordingly, in order to achieve this, one and the other of the terminal connector 150B and the recording connector 220B are modified into a male type and a female type, respectively, so as to be fitted with each other.

Furthermore, the batch processing may be achieved without using the recording medium 300. In order to achieve this, the nurse operation assistance system of a wireless communication type illustrated in FIG. 5 may be modified as follows. First, in the nurse operation assistance system 100B, the transmitter 150 may add, to the input value (identified result), not only the patient ID but also the necessary item and the writing time instant, modulate the carrier using a signal with those additions, and transmit the modulated wave as the transmitted wave to the recording device 200B wirelessly. On the other hand, the recording device 200B may comprise a buffer memory for temporarily storing the identified result (input value) transmitted from the nurse operation assistance terminal 100B without directly supplying it to the recording unit 240. Then, at the suitable time instant after lapse of the above-mentioned time zone, the response speech signals (input values) are successively supplied from the buffer memory to the recording unit 240.

### Third Example Embodiment

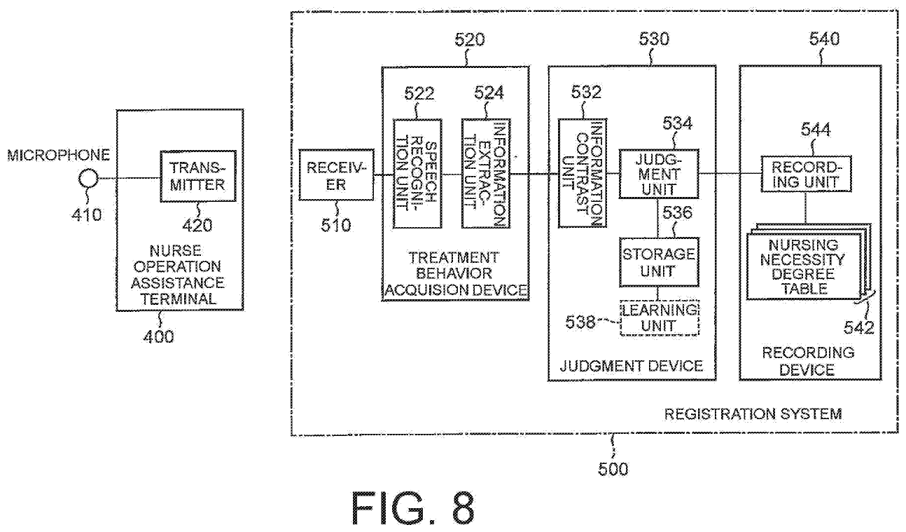

FIG. 8 is a block diagram for illustrating a configuration of a nurse operation assistance system according to a third example embodiment of the present invention. As illustrated in FIG. 8, the nurse operation assistance system comprises a nurse operation assistance terminal 400 and a registration system 500.

In the third example embodiment, the following case is supposed. As is well known, a service pattern of a nurse's operation is classified into day shift, evening shift, and midnight shift.

In the example being illustrated, it is supposed that the specific nurse is in charge of the day shift. Of course, this embodiment is similarly applicable in a case where he/she is in charge of the evening shift or the midnight shift. In the illustrated nurse operation assistance system, a case where the specific nurse treats a plurality of patients of whom he/she is in charge in the day shift is supposed. Since the respective patients are assigned with the patient IDs as described above, there are nursing necessity degree tables which are different for every patient ID. In addition, when the specific nurse performs treatments on the plurality of patients of whom he/she is in charge, he/she generally performs the treatments while calling a name of each patient. Accordingly, it is to be noted that the patient ID can be identified by the name.

In addition, the third example embodiment exemplifies a case of entering, in nursing necessity degree tables for the plurality of patients of whom the specific nurse is in charge, the nursing necessity degrees by real-time processing in the middle of the day shift. However, the present invention is not limited to the real-time processing, and is applicable to batch processing which will later be described.

The nurse operation assistance terminal 400 is a nurse operation assistance terminal which can automatically input, at a treatment site, information required for discriminating nursing necessity degrees for the plurality of patients of whom the specific nurse is in charge. In the example being illustrated, the nurse operation assistance terminal 400 is exclusively assigned to the specific nurse. Accordingly, the specific nurse always carries (wears) the nurse operation assistance terminal 400 in question throughout the day shift. However, the nurse operation assistance terminal 400 may not exclusively be assigned to the specific nurse but may be shared among a plurality of nurses. For instance, the next nurse taking over the operation of the specific nurse in question may use the nurse operation assistance terminal 400. The nurse operation assistance terminal 400 comprises a microphone 410 and a transmitter 420.

In a sickroom, the specific nurse generates utterance for each patient (specific patient), of whom he/she is in charge, in conjunction with the treatment behavior performed on the patient. In addition, conversation is exchanged between the specific nurse and the specific patient in conjunction with the treatment behavior performed on the specific patient.

Specifically, as the utterance by the specific nurse, the following is supposed. For instance, it is assumed that the specific patient has a name of “Mr. A”. In addition, it is assumed that the treatment is a “wound treatment”. In this case, the specific nurse utters, to the specific patient, “Mr. A, let me perform the wound treatment”. Of course, for a different patient of whom the specific nurse is in charge, for example, he/she may call the patient “Mr. B” or the like, which is a name of the different patient.

In addition, as the conversation exchanged between the specific nurse and the plurality of patients of whom the specific nurse is in charge, the following is supposed. In this example, it is assumed that first through third specific patients, of whom the specific nurse is in charge, have names of “Mr. A”, “Mr. B”, and “Mr. C”, respectively. It is assumed that a patient's condition is “putting on/off of clothing”.

In this case, as a first example, it is assumed that the specific nurse utters, to the first specific patient, “Mr. A, please put on your clothes”. In response to this utterance, it is supposed that the first specific patient answers “Yes, I do.” while he is performing an operation of putting on his clothes. In this event, since the putting on/off of clothing is judged as “no assistance”, the score is equal to zero.

As a second example, it is assumed that the specific nurse utters, to the second specific patient, “Mr. B, please raise your hands because I will let you put on your clothes”. In response to this utterance, it is supposed that the second specific patient answers “Yes, I do.” while he is performing an operation of raising his hands. In this event, since putting on/off of clothing is judged as “partial assistance”, the score is equal to one.

As a third example, it is assumed that the specific nurse utters, to the third specific patient, “Mr. C, please raise your hands because I will let you put on your clothes. Can you do it?”. It is assumed that the third specific patient is incidentally in a bad condition at that time instant. In such a situation, it is supposed that, in response to this utterance, the third specific patient answers as “Sony, I cannot do it because I'm sick today”. In this event, since the putting on/off of clothing is judged as “total assistance”, the score is equal to two.

The microphone 410 collects such utterances or conversations. Accordingly, the microphone 410 serves as a collection unit configured to collect the utterance generated by the specific nurse to each specific patient and/or conversation exchanged between the specific nurse and each specific patient, in conjunction with a treatment behavior performed on each specific patient of whom the specific nurse is in charge.

The microphone 410 converts a collected speech into a collected speech signal being an electric signal. The converted collected speech signal is sent to the transmitter 420. The transmitter 420 modulates a carrier with the collected speech signal, and transmits a modulated wave as a transmitted wave to the registration system 500 wirelessly. Accordingly, the transmitter 420 serves as an output means configured to output the collected speech signal indicative of the collected speech to the outside.

In the example being illustrated, the nurse operation assistance terminal 400 and the registration system 500 are wirelessly connected to each other using Bluetooth, WiFi, or the like. However, a wireless connection method is not limited thereto.

The registration system 500 receives the collected speech signal transmitted from the nurse operation assistance terminal 400. The registration system 500 comprises a receiver 510, a treatment behavior acquisition device 520, a judgment device 530, and a recording device 540.

The receiver 510 receives, as a received wave, the transmitted wave transmitted from the transmitter 420 of the nurse operation assistance terminal 400. The receiver 510 modulates the received wave to reproduce the collected speech signal. The reproduced collected speech signal is supplied to the treatment behavior acquisition device 520.

The treatment behavior acquisition device 520 acquires treatment behavior information from the collected speech signal as will later be described. The judgment device 530 judges, based on the treatment behavior information, a nursing necessity degree for each patient to produce a judged value, as will later be described. The recording device 540 records the judged value as will later be described.

The treatment behavior acquisition device 520 comprises a speech recognition unit 522 and an information extraction unit 524. The speech recognition unit 522 recognizes the collected speech signal to produce a recognized result as a speech identified result.

Accordingly, the speech recognition unit 522 serves as a speech identification means configured to identify the collected speech signal to produce the speech identified result. The information extraction unit 524 extracts the above-mentioned treatment behavior information from the speech identified result.

Herein, the treatment behavior information includes, for example, in the cases of the examples described above, text data such as “Mr. A”, “wound treatment”, “let me”, “please put on your clothes”, “yes”, “I do”, “I will let you put on your clothes”, “please raise your hands”, “can you do it?”, “sorry”, “I'm sick today”, “I cannot do it”, and so on.

The recording device 540 comprises a plurality of nursing necessity degree tables 542 for recording nursing necessity degrees for every patient and a recording unit 544. Each nursing necessity degree table 542 is assigned with a patient ID. The recording unit 544 automatically records, in a corresponding predetermined field on the nursing necessity degree table 542 in question, the judged result which is sent from the judgment device 530 as will later be described.

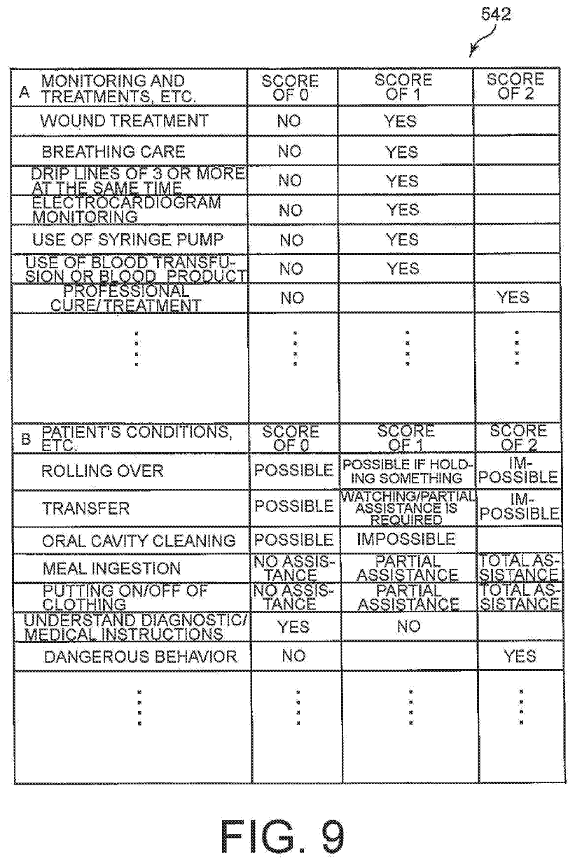

FIG. 9 is a view for illustrating a specific example of each nursing necessity degree table 542. The illustrated nursing necessity degree table 542 includes a plurality of items related to A: monitoring and treatments, etc. and a plurality of items related to B: patient's conditions, etc. For the respective items of the monitoring and treatments, etc., a score of zero is given in a case of “no” and a score of one or two is given in a case of “yes”. On the other hand, for the respective items of the patient's conditions, etc., for example, “can”, “cannot,” or the like is presented and a score of zero, one, or two is given.

Turning back to FIG. 8, the judgment device 530 comprises an information contrast unit 532, a judgment unit 534, and a storage unit 536. In the example being illustrated, the judgment device 530 further comprises a learning unit 538, as shown in a broken line. However, the learning unit 538 is not always necessary in the third example embodiment and may be provided optionally. In other words, since the learning unit 538 is a device for learning and generating a judgment rule as will later be described, the learning unit may be a separate unit from the judgment device 530 and may save only a result thereof (judgment rule) in the storage unit 536.

The information contrast unit 532 contrasts the treatment behavior information with predetermined necessary items to produce a plurality of keywords required to specify the nursing necessity degree. Herein, the keywords may be, for example, “wound”, “drip”, “injection”, “meal”, “transfer”, “transportation”, “clothing”, and so on, which are described in respective fields of the nursing necessity degree table 542 in FIG. 9.

The storage unit 536 saves the judgment rule generated by the learning unit 538 as will later be described.

The judgment unit 534 judges the nursing necessity degree for every patient based on the judgment rule saved in the storage unit 536, using a combination of the plurality of keywords as will later be described, and produces the judged value.

Next, description will proceed to a specific example of judgment in the judgment unit 534.

First, as the nursing necessity degree, a case of “wound treatment” will be described by way of example. It is assumed that the judgment unit 534 is supplied from the information contract unit 532 with a combination of keywords of “wound”, “treatment”, and “do”. In this event, the judgment unit 534 judges that the wound treatment is “yes” and produces the judged value indicative of that effect. In this case, the recording unit 544 of the recording device 540 records, based on the judged value, “yes” in a field of “wound treatment” in the nursing necessity degree table 542 of the patient ID in question in FIG. 9.

Next, as the nursing necessity degree, a case of “putting on/off of clothing” will be described by way of example. At first, it is assumed that the judgment unit 534 is supplied from the information contrast unit 532 with a combination of keywords of “clothes” and “please put on.” In this event, the judgment unit 534 judges the putting on/off of clothing as “no assistance” and produces the judged value indicative of that effect. In this case, the recording unit 544 of the recording device 540 records, based on the judged value, “no assistance” in a field of “putting on/off of clothing” in the nursing necessity degree table 542 of the patient ID in question in FIG. 9.

Next, it is assumed that the judgment unit 534 is supplied from the information contrast unit 532 with a combination of keywords of “clothes”, “raise your hands”, and “yes”. In this event, the judgment unit 534 judges the putting on/off of clothing as “partial assistance” and produces the judged value indicative of that effect. In this case, the recording unit 544 of the recording device 540 records, based on the judged value, “partial assistance” in a field of “putting on/off of clothing” in the nursing necessity degree table 542 of the patient ID in question in FIG. 9.

In addition, it is assumed that the judgment unit 534 is supplied from the information contrast unit 532 with a combination of keywords of “clothes”, “raise your hands”, and “I cannot do”. In this event, the judgment unit 534 judges the putting on/off of clothing as “total assistance” and produces the judged value indicative of that effect. In this case, the recording unit 544 of the recording device 540 records, based on the judged value, “total assistance” in the field of “putting on/off of clothing” in the nursing necessity degree table 542 of the patient ID in question in FIG. 9.

Furthermore, it is assumed that the judgment unit 534 is supplied from the information contrast unit 532 with a particular keyword. In this event, the judgment unit 534 refers to an ordering system (not shown) to judge a score. Herein, the “ordering system” means a system for directly inputting, to a computer, contents of orders given from doctors to medical technicians, such as nurses or pharmacists, to accurately and quickly transmit the contents to respective sections.

For instance, it is assumed that the judgment unit 534 is supplied from the information contrast unit 532 with a keyword of “drip” as the particular keyword. In this event, the judgment unit 534 refers to the ordering system to judge whether or not the number of drips is three or more. When a signal indicative of “yes” is sent from the ordering system in this inquiry, the judgment unit 534 judges three or more drip lines at the same time as “yes” and produces the judged value indicative of that effect. In this case, the recording unit 544 of the recording device 540 records, based on the judged value, “yes” in a field of “three or more drip lines at the same time” in the nursing necessity degree table 542 of the patient ID in question in FIG. 9. The score at this time is equal to one.

On the other hand, it is assumed that a signal indicative of “no” is sent from the ordering system for the inquiry to the above-mentioned ordering system. In this event, the judgment unit 534 judges the drip lines of three or more at the same time as “no” and produces the judged value indicative of that effect. In this case, the recording unit 544 of the recording device 540 records, based on the judged value, “no” in the field of “drip lines of three or more at the same time” in the nursing necessity degree table 542 of the patient ID in question in FIG. 9. A score at that time is zero.

The learning unit 538 is supplied with the plurality of keywords and, using, as teaching signals, data indicative of treatment behaviors actually performed in conjunction with a plurality of utterances generated from each of a plurality of nurses to each of a plurality of patients and/or a plurality of conversations exchanged between each of the plurality of nurses and each of the plurality of patients, performs machine learning to generate the judgment rule.

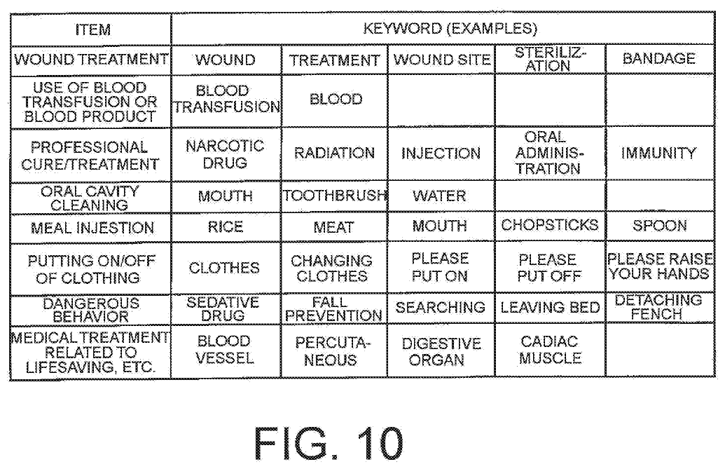

FIG. 10 is a view for illustrating an example of training data used on machine-learning by the learning unit 538. For instance, it is understood that, when “wound”, “treatment”, “wound site”, “sterilization”, “bandage”, and so on are inputted as the keywords, data indicative of a treatment behavior of “wound treatment” is used as the teaching signal. Alternatively, it is understood that, when “mouth”, “toothbrush”, “water”, and so on are inputted as the keywords, data indicative of a treatment behavior of “oral cavity cleaning” are used as the teaching signal.

In addition, a machine learning technique used in the learning unit 538 is not especially limited. For instance, a regression technique such as SVM (Support Vector Machine), a clustering technique such as a k-nearest neighbor algorithm, and a neural network technique may be used as the machine learning technique.

Now, effects of the third example embodiment will be described. As described above, the nurse operation assistance terminal 400 according to the third example embodiment can automatically input, at the treatment site, the information required to judge the nursing necessity degrees for the plurality of patients of whom the specific nurse is in charge (i.e. collected speeches obtained by collecting the utterances or the conversations of the specific nurse). As a result, it is possible to reliably eliminate a difference in judgment among individuals and the mistaken memory of the nurse. Furthermore, in the registration system 500, the judgment device 530 judges the nursing necessity degree for every patient and the recording device 540 automatically enters it in the nursing necessity degree table 542 of the patient ID in question. Accordingly, it is possible to reduce the operation of entering the nursing necessity degree by the nurse using the user terminal that is carried out in the related art.

[Operation of Nurse Operation Assistance System]

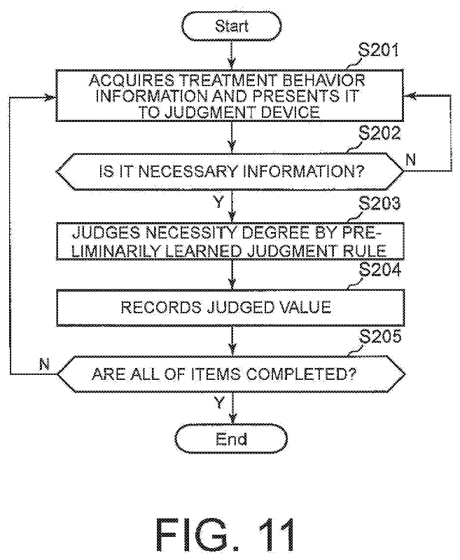

FIG. 11 is a flow chart for illustrating a flow of operation of the nurse operation assistance system illustrated in FIG. 8. Now, the flow of the operation of the nurse operation assistance system will be described with reference to FIGS. 8 and 11.

First, in the nurse operation assistance terminal 400, the microphone 410 collects, as the collected speech, the utterance generated by the specific nurse and/or the conversation exchanged between the specific nurse and each patient of whom the specific nurse is in charge, and the transmitter 420 transmits the transmitted wave indicative of the collected speech signal.

In the registration system 500, at first, the receiver 510 receives the transmitted wave as the received wave to reproduce the collected speech signal. The treatment behavior acquisition device 520 acquires the treatment behavior information from the collected speech signal to present it to the judgment device 530 (Step S201).

The information contrast unit 532 of the judgment device 530 contrasts the treatment behavior information with the necessary items and judges whether or not there is the necessary information (keyword) (Step S202). If there is the necessary information (keyword) (Y in Step S202), the judgment unit 534 of the judgment device 530 judges the necessity degree using the preliminarily learned judgment rule to produce the judged value (Step S203).

The recording unit 544 of the recording device 540 records the judged value in the corresponding field in the nursing necessity degree table 542 of the patient ID in question (Step S204).

The nurse operation assistance system repeats the above-mentioned processing (Steps S201 to S204) until all of items in the nursing necessity table 542 are completed (Step S205).

### Third Modification

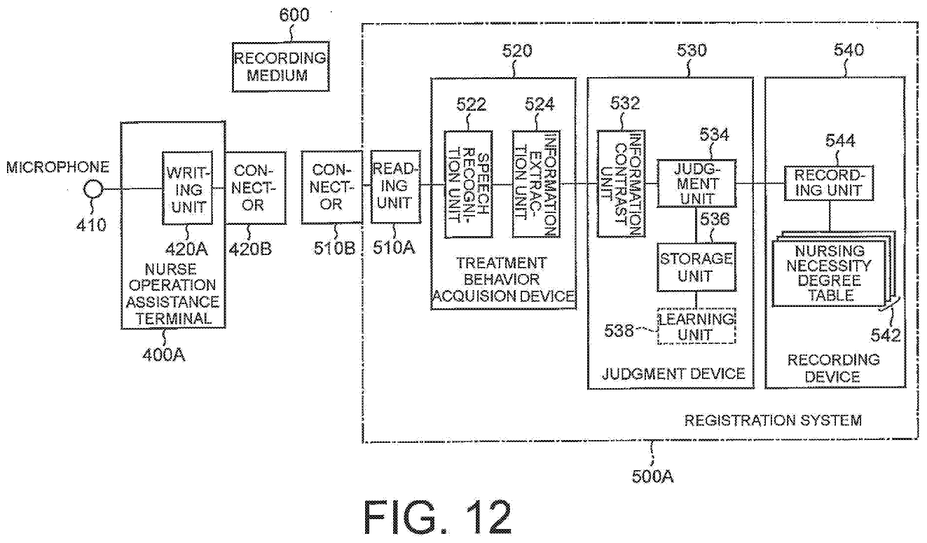

Next, referring to FIG. 12, description will proceed to a nurse operation assistance system according to a modification (third modification) of the third example embodiment. Although the case of the real-time processing is described in the above-mentioned third example embodiment by way of example, batch processing as will be described hereafter may be applied.

In a case of the batch processing, a recording medium 600, such as a memory stick or a USB memory, is used. Such a recording medium is prepared for every nurse. Accordingly, the illustrated recording medium 600 is a recording medium for the specific nurse. However, the recording medium 600 may be shared among a plurality of nurses. For instance, one recording medium 600 may be shared among three nurses who successively take over their operations on the day shift, the evening shift, and the midnight shift.

In addition, in the nurse operation assistance system according to the third modification, the nurse operation assistance terminal 400 and the registration system 500 illustrated in FIG. 8 are changed into a nurse operation assistance terminal 400A and a registration system 500A, respectively, which will be described in the following. Specifically, the nurse operation assistance terminal 400A uses a writing unit 420A and a terminal connector 420B in lieu of the transmitter 420. The writing unit 420A is inserted and connected between the terminal connector 420B and the microphone 410. On the other hand, the registration system 500A uses a reading unit 510A and a registration connector 510B in lieu of the receiver 510. The reading unit 510A is inserted and connected between the registration connector 510B and the treatment behavior acquisition device 520.

First, the above-mentioned recording medium 600 for the specific nurse is inserted into the terminal connector 420B of the nurse operation assistance terminal 400A during a period of time when the specific nurse in question performs the operation (in a case of this example, a time zone of the day shift). In this state, the collected speech signal collected by the microphone 410 is sent to the writing unit 420A. The writing unit 420A adds a collection time instant to the collected speech signal and writes the added information as recorded information in the recording medium 600 for the specific nurse via the terminal connector 420B. Herein, it is noted that the patient ID need not be added to the recorded information, as is different from a case of the above-mentioned first modification. This is because the name of the patient (e.g. the “Mr. A”) is included in the utterance generated by the specific nurse and the patient ID can be identified based on the name of the patient.

The recording medium 600 for the specific nurse, with the above-mentioned recorded information written therein, is inserted into the registration connector 510B of the registration system 500A at a time instant which is a predetermined time interval (e.g. 30 minutes) prior to a time instant when the operation (day shift) of the specific nurse is finished. In the registration system 500A, the reading unit 510A reads, out of the recording medium 600 for the specific nurse, the recorded information written therein via the registration connector 510B. The read-out recorded information is supplied to the treatment behavior acquisition device 520. Since the read-out recorded information includes the information (names) for identifying the plurality of patients of whom the specific nurse is in charge, the judgment device 530, which receives the treatment behavior information from the treatment behavior acquisition device 520, can judge the nursing necessity degree for every patient ID.

As described above, the batch processing is carried out. The third modification also has effects similar to those of the above-mentioned third example embodiment. That is, in the third modification also, it is possible to reliably eliminate a difference in judgment among individuals and mistaken memory of the nurse and to reduce the entering operation.

Although the recording medium 600 is provided outside in the above-mentioned third modification, the recording medium 600 may be installed inside the nurse operation assistance terminal 400A. In this event, it is necessary for the terminal connector 420B of the nurse operation assistance terminal 400A to be directly connected to the registration connector 510B of the registration system 500A at the time instant which is the predetermined time interval prior to the time instant when the operation (day shift) of the specific nurse is finished. Accordingly, in order to achieve this, one and the other of the terminal connector 420B and the registration connector 510B are modified into a male type and a female type, respectively, so as to be fitted with each other.

Furthermore, the batch processing may be achieved without using the recording medium 600. In order to achieve this, the nurse operation assistance system of a wireless communication type illustrated in FIG. 8 may be modified as follows. First, in the nurse operation assistance system 400, the transmitter 420 may add the collection time instant to the collected speech signal, modulate the carrier using the signal with the addition, and transmit the modulated wave as the transmitted wave to the registration system 500 wirelessly. On the other hand, the registration system 500 may comprise a buffer memory for temporarily storing the collected speech signal transmitted from the nurse operation assistance terminal 400 without directly supplying it to the treatment behavior acquisition device 520. Then, at the time instant which is the predetermined time interval prior to the time instant when the operation (day shift) of the specific nurse is finished, the collected speech signals are successively supplied from the buffer memory to the treatment behavior acquisition device 520.

### Fourth Example Embodiment

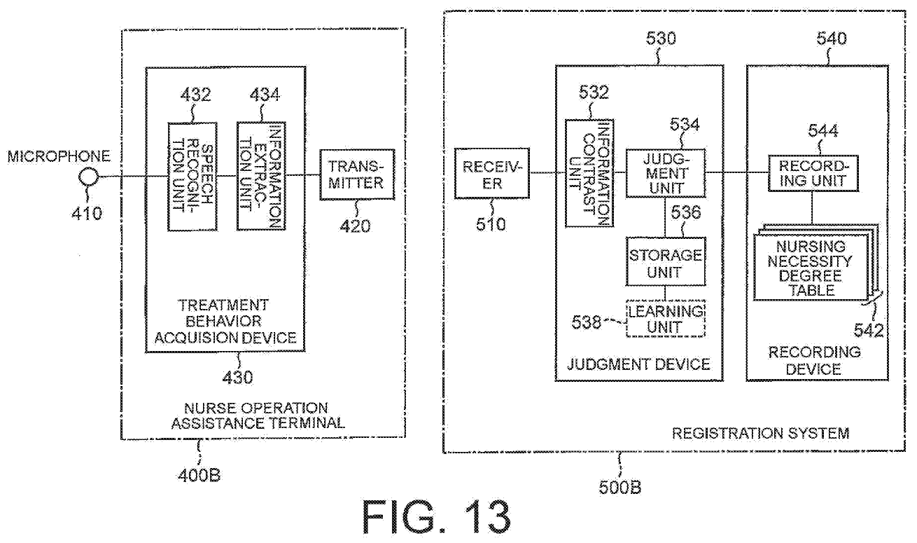

FIG. 13 is a block diagram for illustrating a configuration of a nurse operation assistance system according to a fourth example embodiment of the present invention. As illustrated in FIG. 13, the nurse operation assistance system comprises a nurse operation assistance terminal 400B and a registration system 500B.

In the fourth example embodiment also, a case similar to that of the above-mentioned third example embodiment is supposed. That is, in the illustrated nurse operation assistance system, a case where the specific nurse treats a plurality of patients of whom the specific nurse is in charge in his/her duty time zone (day shift) is supposed.

In addition, like in the third example embodiment, the fourth example embodiment also exemplifies a case of entering the nursing necessity degrees for the plurality of patients of whom the specific nurse is in charge by real-time processing. However, the present invention is not limited to the real-time processing, and is applicable to batch processing also.

A difference between the above-mentioned third example embodiment and the fourth example embodiment exists in a signal which is transmitted and received between the nurse operation assistance terminal and the registration system. In detail, in the above-mentioned third example embodiment, the collected speech signals collected by the microphone are transmitted and received. On the other hand, in the fourth example embodiment, the treatment behavior information is transmitted and received, as will be later described.

The nurse operation assistance terminal 400B is similar in structure and operation to the nurse operation assistance terminal 400 illustrated in FIG. 8 except that a treatment behavior acquisition device 430 is further provided. That is, the nurse operation assistance terminal 400B comprises the microphone 410, the transmitter 420, and the treatment behavior acquisition device 430.

The treatment behavior acquisition device 430 is inserted between the microphone 410 and the transmitter 420. The treatment behavior acquisition device 430 acquires the treatment behavior information from the collected speech signal collected by the microphone 410. The treatment behavior acquisition device 430 comprises a speech recognition unit 432 and an information extraction unit 434. The speech recognition unit 422 identifies the collected speech signal to produce a speech identified result. The information extraction unit 434 extracts the treatment behavior information from the speech identified result.

The treatment behavior information is supplied to the transmitter 420. Accordingly, the transmitter 420 modulates a carrier using the treatment behavior information, and transmits the modulated wave to the registration system 500B as the transmitted wave wirelessly.

In the example being illustrated also, the nurse operation assistance terminal 400B and the registration system 500B are wirelessly connected to each other using Bluetooth, WiFi, or the like. However, a wireless connection method is not limited thereto.

The registration system 500B is similar in structure and operation to the registration system 500 illustrated in FIG. 8 except that the treatment behavior acquisition device 520 is deleted. That is, the registration system 500B comprises the receiver 510, the judgment device 530, and the recording device 540. The receiver 510 and the judgment device 530 are directly connected to each other.

The receiver 510 receives the transmitted wave transmitted from the transmitter 420 of the nurse operation assistance terminal 400B as a received wave. The receiver 510 demodulates the received wave to reproduce the above-mentioned treatment behavior information. The reproduced treatment behavior information is supplied to the judgment device 530. The judgment device 530 judges, based on the treatment behavior information, the patient necessity degree for every patient ID to produce the judged value. The recording unit 544 of the recording device 540 automatically records the judged value in a corresponding predetermined field on the patient necessity degree table 542 for every patient ID.

In addition, the nursing necessity table 542 stores contents illustrated in FIG. 9 in the manner similar to the above-mentioned third example embodiment.

Now, effects of the fourth example embodiment will be described. As described above, the nurse operation assistance terminal 400B according to the fourth example embodiment can automatically input, at the treatment site, the information required to discriminate the nursing necessity degrees for the plurality of patients of whom the specific nurse is in charge (i.e. the treatment behavior information). As a result, it is possible to reliably eliminate a difference in judgment among individuals and the mistaken memory of the nurse. Furthermore, in the registration system 500B, the judgment device 530 judges the nursing necessity degree for every patient and the recording device 540 automatically enters it in the nursing necessity degree table 542 of the patient ID in question. Accordingly, it is possible to reduce the operation of entering the nursing necessity degree by the nurse using the user terminal that is carried out in the related art.

[Operation of Nurse Operation Assistance System]

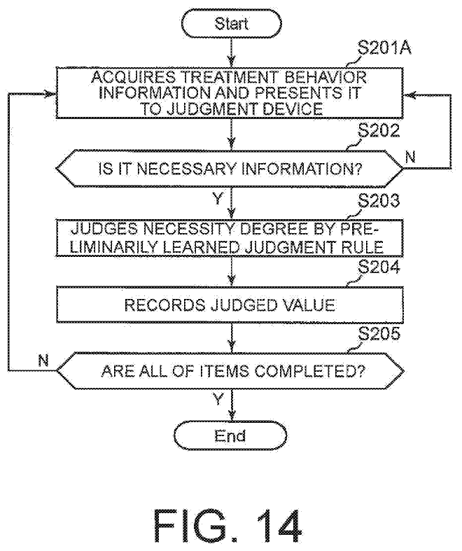

FIG. 14 is a flow chart for illustrating a flow of operation of the nurse operation assistance system illustrated in FIG. 13. Now, the flow of the operation of the nurse operation assistance system will be described with reference to FIGS. 13 and 14.

First, in the nurse operation assistance terminal 400B, the microphone 410 collects, as the collected speech, the utterance generated by the specific nurse and/or the conversation exchanged between the specific nurse and the respective patients of whom the specific nurse is in charge. The treatment behavior acquisition device 430 acquires the treatment behavior information from the collected speech signal indicative of the collected speech. The treatment behavior information is presented to the judgment device 530 through the transmitter 420 and the receiver 510 (Step S201A).

The information contrast unit 532 of the judgment device 530 contrasts the treatment behavior information with the necessary items and judges whether or not there is the necessary information (keyword) (Step S202). If there is the necessary information (keyword) (Y in Step S202), the judgment unit 530 of the judgment device 530 judges the necessity degree using the preliminarily learned judgment rule to produce the judged value (Step S203).

The recording unit 544 of the recording device 540 records the judged value in the corresponding field in the nursing necessity degree table 542 of the patient ID in question (Step S204).

The nurse operation assistance system repeats the above-mentioned processing (Steps S201A to S204) until all of items in the nursing necessity table 542 are completed (Step S205).

### Fourth Modification

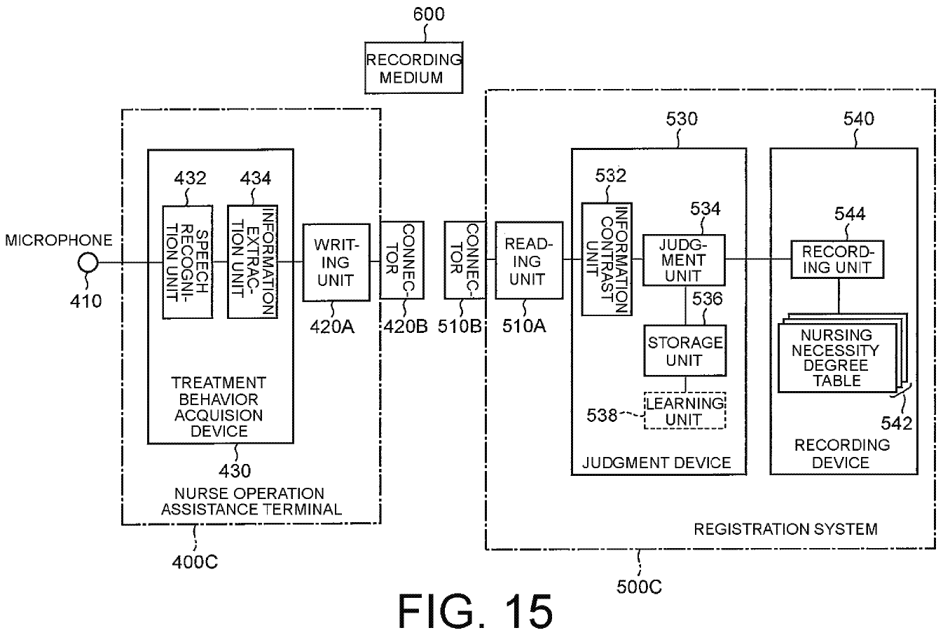

Next, referring to FIG. 15, description will proceed to a nurse operation assistance system according to a modification (fourth modification) of the fourth example embodiment. Although the case of the real-time processing is described in the above-mentioned fourth example embodiment by way of example, batch processing as will be described hereafter may be applied.

In a case of the batch processing, a recording medium 600, such as a memory stick or a USB memory, is used. Such a recording medium is prepared for every nurse. Accordingly, the illustrated recording medium 600 is a recording medium for the specific nurse. However, the recording medium 600 may be shared among a plurality of nurses, as mentioned above. For instance, one recording medium 600 may be shared among three nurses who successively take over their operations on the day shift, the evening shift, and the midnight shift.

In addition, in the nurse operation assistance system according to the fourth modification, the nurse operation assistance terminal 400B and the registration system 500B illustrated in FIG. 13 are changed into a nurse operation assistance terminal 400C and a registration system 500C, respectively, which will be described in the following. Specifically, the nurse operation assistance terminal 400C uses the writing unit 420A and the terminal connector 420B in lieu of the transmitter 420. The writing unit 420A is inserted and connected between the terminal connector 420B and the treatment behavior acquisition device 430. On the other hand, the registration system 500C uses the reading unit 510A and the registration connector 510B in lieu of the receiver 510. The reading unit 510A is inserted and connected between the registration connector 510B and the judgment device 530.

First, the above-mentioned recording medium 600 for the specific nurse is inserted into the terminal connector 420B of the nurse operation assistance terminal 400C during a period of time when the specific nurse in question performs the operation (in a case of this example, a time zone of the day shift). In this state, the treatment behavior information supplied from the treatment behavior acquisition device 430 is sent to the writing unit 420A. The writing unit 420A adds a collection time instant to the treatment behavior information and writes the added information as recorded information in the recording medium 600 for the specific nurse via the terminal connector 420B. Herein, it is noted that the patient ID need not be added to the recorded information, as is different from a case of the above-mentioned second modification. This is because the name of the patient (e.g. the “Mr. A”) is included in the utterance generated by the specific nurse and the patient ID can be identified based on the name of the patient.

The recording medium 600 for the specific nurse, with the above-mentioned recorded information written therein, is inserted into the registration connector 510B of the registration system 500C at a time instant which is a predetermined time interval (e.g. thirty minutes) prior to a time instant when the operation (day shift) of the specific nurse is finished. In the registration system 500C, the reading unit 510A reads, out of the recording medium 600 for the specific nurse, the recorded information written therein via the registration connector 510B. The read-out recorded information is supplied to the judgment device 530. Since the read-out recorded information includes the information (names) for identifying the plurality of patients of whom the specific nurse is in charge, the judgment device 530, which receives the treatment behavior information, can judge the nursing necessity degree for every patient ID.

As described above, the batch processing is carried out. The fourth modification also has effects similar to those of the above-mentioned fourth example embodiment. That is, in the fourth modification also, it is possible to reliably eliminate a difference in judgment among individuals and mistaken memory of the nurse and to reduce the entering operation.

Although the recording medium 600 is provided outside in the above-mentioned fourth modification, the recording medium 600 may be installed inside the nurse operation assistance terminal 400C. In this event, it is necessary for the terminal connector 420B of the nurse operation assistance terminal 400C to be directly connected to the registration connector 510B of the registration system 500C at the time instant which is the predetermined time interval prior to the time instant when the operation (day shift) of the specific nurse is finished. Accordingly, in order to achieve this, one and the other of the terminal connector 420B and the registration connector 510B are modified into a male type and a female type, respectively, so as to be fitted with each other.

Furthermore, the batch processing may be achieved without using the recording medium 600. In order to achieve this, the nurse operation assistance system of a wireless communication type illustrated in FIG. 13 may be modified as follows. First, in the nurse operation assistance system 400B, the transmitter 420 may add the collection time instant to the treatment behavior information, modulate the carrier using the signal with the addition, and transmit the modulated wave as the transmitted wave to the registration system 500B wirelessly. On the other hand, the registration system 500B may comprise a buffer memory for temporarily storing the treatment behavior information transmitted from the nurse operation assistance terminal 400B without directly supplying it to the judgment device 530. Then, at the time instant which is the predetermined time interval prior to the time instant when the operation (day shift) of the specific nurse is finished, the treatment behavior information is successively supplied from the buffer memory to the judgment device 530.

[Hardware Configuration of Registration System]

The registration systems 500 to 500C mentioned above may be implemented by hardware or may be implemented by software. In addition, the registration systems 500 to 500C may be implemented by a combination of hardware and software.

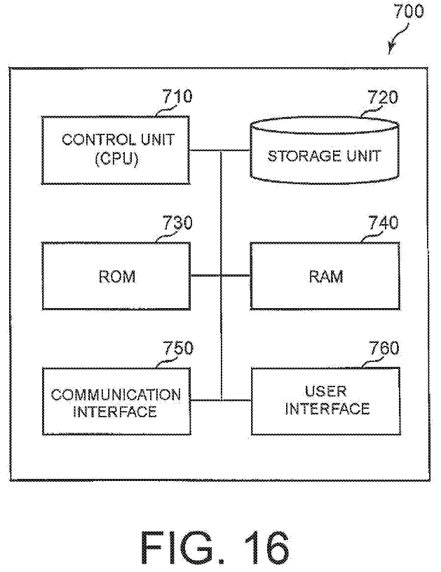

FIG. 16 is a block diagram for illustrating one example of an information processing apparatus (computer) constituting the registration systems 500 to 500C.

As shown in FIG. 16, the information processing apparatus 700 comprises a control unit (CPU: Central Processing Unit) 710, a storage unit 720, an ROM (Read Only Memory) 730, an RAM (Random Access Memory) 740, a communication interface 750, and a user interface 760.

The control unit (CPU) 710 may implement various functions of the registration systems 500 to 500C by developing and executing, in the RAM 740, a program which is stored in the storage unit 720 or the ROM 730. In addition, the control unit (CPU) 710 may comprise an internal buffer which is adapted to temporarily store data or the like.

The storage unit 720 comprises a bulk storage medium which can hold various types of data and may be implemented by a storage medium such as an HDD (Hard Disk Drive) and an SSD (Solid State Drive). The storage unit 720 may be a cloud storage existing in a communication network when the information processing apparatus 700 is connected to the communication network via the communication interface 750. The storage unit 720 may hold the program readable by the control unit (CPU) 710.

The ROM 730 is a nonvolatile storage device which may comprise a flash memory having a small capacity as compared to the storage unit 720. The ROM 730 may hold a program which is readable by the control unit (CPU) 710. The program readable by the control unit (CPU) 710 may be held in at least one of the storage unit 720 and the ROM 730.

The program readable by the control unit (CPU) 710 may be supplied to the information processing apparatus 700 in a state where it is non-transitorily stored in various storage media readable by the computer. Such storage media include, for example, a magnetic tape, a magnetic disk, a magneto-optical disc, a CD-ROM (Compact Disc-Read Only Memory), a CD-R (Compact Disc-Readable), a CD-RW (Compact Disc-ReWritable), and a semiconductor memory.

The RAM 740 comprises a semiconductor memory such as a DRAM (Dynamic Random Access Memory) and an SRAM (Static Random Access Memory) and may be used as an internal buffer which temporarily stores data and so on.

The communication interface 750 is an interface which connects the information processing system 700 and the communication network via wire or wirelessly.

The user interface 760 comprises, for example, a display unit such as a display and an input unit such as a keyboard, a mouse, and a touch panel. However, the user interface 760 may not be provided merely for the purpose of implementing the functions of the registration systems 500 to 500C.

While the present invention has been described with reference to the example embodiments thereof, the present invention is not limited thereto. For example, the present invention includes a mode obtained by appropriately combining a part or a whole of the example embodiments described so far or a mode obtained by appropriately modifying those modes.

For example, the recording device internally has (comprises) the nursing necessity degree table in the example embodiments mentioned above and in the modifications mentioned above. However, the nursing necessity degree table may be saved separately from the recording device, for example, in a database of a control center (server) which is connected to the recording device via wire or wirelessly. In a case of such a mode, the recording device or the registration system serves as a user terminal without an input unit and a display unit. In other words, a combination of the nurse operation assistance terminal and the recording device or a combination of the nurse operation assistance terminal and the registration device serves as the user terminal.

In addition, in the batch processing of the third and the fourth modifications mentioned above, registration of the nursing necessity degree by the registration system 500A or 500C is carried out just before the specific nurse finishes the operation (more specifically, at the time instant which is the predetermined time interval prior to the time instant when the operation of the specific nurse is finished). However, the present invention is not limited thereto. For example, in a case where the nurse operation assistance terminal 400A or 400C is shared among three nurses taking over their operations from one to another on the day shift, the evening shift, and the midnight shift, the registration system 500A or 500C may register the nursing necessity degree just before one-day's operation is finished (more specifically, at the time instant which is the predetermined time interval prior to the time instant when the one-day's operation is finished).

A part or a whole of the example embodiments described above may also be described as the following supplementary notes without being limited thereto.

(Supplementary Note 1)

A nurse operation assistance terminal comprising:

a presentation unit configured to sequentially present, to a nurse at a treatment site, necessary items with which a patient is to be treated; and

an input unit configured to input response speeches uttered by the nurse in response to the presented necessary items and to output response speech signals indicative of the response speeches as input values.

(Supplementary Note 2)

The nurse operation assistance terminal according to Supplementary Note 1, wherein the presentation unit comprises a speech notification means configured to notify the nurse of the necessary items with question speeches.

(Supplementary Note 3)

The nurse operation assistance terminal according to Supplementary Note 1 or 2, wherein the input unit comprises a speech identification means configured to identify the inputted response speeches and to output the input values as identified results being text data.

(Supplementary Note 4)

A nurse operation assistance system comprising:

the nurse operation assistance terminal described in Supplementary Note 1 or 2; and

a recording device configured to receive the input values from the nurse operation assistance terminal,
wherein the recording device comprises:

a nursing necessity degree table configured to record the nursing necessity degree;

a speech identification means configured to identify the input values being response speech signals as identified results being text data; and

a recording unit configured to automatically record the identified results in corresponding predetermined fields on the nursing necessity degree table.

(Supplementary Note 5)

A nurse operation assistance system comprising:

the nurse operation assistance terminal described in Supplementary Note 3; and

a recording device configured to receive the identified results from the nurse operation assistance terminal,
wherein the recording device comprises:

a nursing necessity degree table configured to record the nursing necessity degree; and

a recording unit configured to automatically record the identified results in corresponding predetermined fields on the nursing necessity degree table.

(Supplementary Note 6)

A nurse operation assistance terminal comprising:

a collection unit configured to collect, as a collected speech, utterance generated by a nurse to a patient and/or conversation exchanged between the nurse and the patient in conjunction with a treatment behavior performed on the patient by the nurse at a treatment site; and

an output means configured to output a collected speech signal indicative of the collected speech.

(Supplementary Note 7)

A nurse operation assistance system comprising:

the nurse operation assistance terminal described in Supplementary Note 6; and

a registration system configured to receive the collected speech signal from the nurse operation assistance terminal,
wherein the registration system comprises:

a treatment behavior acquisition device configured to acquire treatment behavior information from the collected speech signal;

a judgment device configured to judge a nursing necessity degree based on the treatment behavior information to produce a judged value; and

a recording device configured to record the judged value.

(Supplementary Note 8)

The nurse operation assistance system according to Supplementary Note 7,
wherein the treatment behavior acquisition device comprises:

a speech identification means configured to identify the collected speech signal to produce a speech identified result; and

an information extraction means configured to extract the treatment behavior information from the speech identified result.

(Supplementary Note 9)

The nurse operation assistance system according to Supplementary Note 7 or 8,
wherein the recording device comprises:

a nursing necessity degree table configured to record the nursing necessity degree; and

a recording unit configured to automatically record the judged value in a corresponding predetermined field on the nursing necessity degree table.

(Supplementary Note 10)

The nurse operation assistance system according to any one of Supplementary Notes 7 to 9,
wherein the judgment device comprises:

an information contrast means configured to contrast the treatment behavior information with predetermined necessary items to produce a plurality of keywords required to specify the nursing necessity degree; and

a judgment means configured to judge the nursing necessity degree, based on a judgment rule and using a combination of the plurality of keywords, to produce the judged value.

(Supplementary Note 11)

The nurse operation assistance system according to Supplementary Note 10,
wherein the judgment device further comprises:

a learning unit configured to carry out machine-learning, responsive to the plurality of keywords and using, as teaching signals, data indicative of treatment behaviors actually performed in conjunction with a plurality of utterances generated by a plurality of nurses to a plurality of patients and/or a plurality of conversations exchanged between the plurality of nurses and the plurality of patients, to generate the judgment rule; and

a storage unit configured to store the generated judgment rule.

(Supplementary Note 12)

A nurse operation assistance method, comprising:

acquiring, by a treatment behavior acquisition device, treatment behavior information from a collected speech signal received from a nurse operation assistance terminal;

contrasting, by an information contract means, the treatment behavior information with predetermined necessary items to produce a plurality of keywords required to specify a nursing necessity degree;

judging, by a judgment means, the nursing necessity degree, based on a judgment rule and using a combination of the plurality of keywords, to produce a judged value; and

automatically recording, by a recording unit, the judged value in a corresponding predetermined field on a nursing necessity degree table.

(Supplementary Note 13)

The nurse operation assistance method according to Supplementary Note 12, further comprising:

carrying out machine-learning, by a learning unit, responsive to the plurality of keywords and using, as teaching signals, data indicative of treatment behaviors actually performed in conjunction with a plurality of utterances generated by a plurality of nurses to a plurality of patients and/or a plurality of conversations exchanged between the plurality of nurses and the plurality of patients, to generate the judgment rule; and

storing, by a storage unit, the generated judgment rule.

(Supplementary Note 14)

A recording medium for storing a nurse operation assistance program which causes a computer to execute:

a treatment behavior acquisition step of acquiring treatment behavior information from a collected speech signal received from a nurse operation assistance terminal;

an information contrast step of contrasting the treatment behavior information with predetermined necessary items to produce a plurality of keywords required to specify a nursing necessity degree;

a judgment step of judging the nursing necessity degree, based on a judgment rule and using a combination of the plurality of keywords, to produce a judged value; and

a recording step of automatically recording the judged value in a corresponding predetermined field on a nursing necessity degree table.

(Supplementary Note 15)

The nurse operation assistance program recording medium according to Supplementary Note 14, wherein the nurse operation assistance program causes the computer to further execute:

a learning step of carrying out machine-learning, responsive to the plurality of keywords and using, as teaching signals, data indicative of treatment behaviors actually performed in conjunction with a plurality of utterances generated by a plurality of nurses to a plurality of patients and/or a plurality of conversations exchanged between the plurality of nurses and the plurality of patients, to generate the judgment rule; and

a step of storing the generated judgment rule in a storage unit.

(Supplementary Note 16)

A nurse operation assistance terminal comprising

a collection unit configured to collect utterance generated by a nurse to a patient and/or a conversation exchanged between the nurse and the patient in conjunction with a treatment behavior performed on the patient by the nurse and to produce a collected speech signal indicative of a collected speech; and

a treatment behavior acquisition unit configured to acquire treatment behavior information from the collected speech signal.

(Supplementary Note 17)

The nurse operation assistance terminal according to Supplementary Note 16,
wherein the treatment behavior acquisition unit comprises:

a speech identification means configured to identify the collected speech signal to produce a speech identified result; and

an information extraction means configured to extract the treatment behavior information from the speech identified result.

(Supplementary Note 18)

A nurse operation assistance system comprising:

the nurse operation assistance terminal described in Supplementary Note 16 or 17; and

a registration system configured to receive the treatment behavior information from the nurse operation assistance terminal,
wherein the registration system comprises:

a judgment device configured to judge a nursing necessity degree based on the treatment behavior information to produce a judged value; and

a recording device configured to record the judged value.

(Supplementary Note 19)

The nurse operation assistance system according to Supplementary Note 18,
wherein the recording device comprises:

a nursing necessity degree table configured to record the nursing necessity degree; and

a recording unit configured to automatically record the judged value in a corresponding predetermined field on the nursing necessity degree table.

(Supplementary Note 20)

The nurse operation assistance system according to Supplementary Note 18 or 19,
wherein the judgment device comprises:

an information contrast means configured to contrast the treatment behavior information with predetermined necessary items to produce a plurality of keywords required to specify the nursing necessity degree; and

a judgment means configured to judge the nursing necessity degree, based on a judgment rule and using a combination of the plurality of keywords, to produce the judged result.

(Supplementary Note 21)

The nurse operation assistance system according to Supplementary Note 20,
wherein the judgment device further comprises:

a learning unit configured to carry out machine-learning, responsive to the plurality of keywords and using, as teaching signals, data indicative of treatment behaviors actually performed in conjunction with a plurality of utterances generated by a plurality of nurses to a plurality of patients and/or a plurality of conversations exchanged between the plurality of nurses and the plurality of patients, to generate the judgment rule; and a storage unit configured to store the generated judgment rule.

(Supplementary Note 22)

A nurse operation assistance method, comprising:

contrasting, by an information contract means, treatment behavior information received from a nurse operation assistance terminal with predetermined necessary items to produce a plurality of keywords required to specify a nursing necessity degree;

judging, by a judgment means, the nursing necessity degree, based on a judgment rule and using a combination of the plurality of keywords, to produce a judged value; and

automatically recording, by a recording unit, the judged value in a corresponding predetermined field on a nursing necessity degree table.

(Supplementary Note 23)

The nurse operation assistance method according to Supplementary Note 22, further comprising:

carrying out machine-learning, by a learning unit, responsive to the plurality of keywords and using, as teaching signals, data indicative of treatment behaviors actually performed in conjunction with a plurality of utterances generated by a plurality of nurses to a plurality of patients and/or a plurality of conversations exchanged between the plurality of nurses and the plurality of patients, to generate the judgment rule; and

storing, by a storage unit, the generated judgment rule.

(Supplementary Note 24)

A recording medium for storing a nurse operation assistance program which causes a computer to execute:

an information contract step of contrasting treatment behavior information received from a nurse operation assistance terminal with predetermined necessary items to produce a plurality of keywords required to specify a nursing necessity degree;

a judgment step of judging the nursing necessity degree, based on a judgment rule and using a combination of the plurality of keywords, to produce a judged value; and

a recording step of automatically recording the judged value in a corresponding predetermined field on a nursing necessity degree table.

(Supplementary Note 25)

The nurse operation assistance program recording medium according to Supplementary Note 24, wherein the nurse operation assistance program causes the computer to further execute:

a learning step of carrying out machine-learning, responsive to the plurality of keywords and using, as teaching signals, data indicative of treatment behaviors actually performed in conjunction with a plurality of utterances generated by a plurality of nurses to a plurality of patients and/or a plurality of conversations exchanged between the plurality of nurses and the plurality of patients to generate the judgment rule; and

a step of storing the generated judgment rule in a storage unit.

This application is based upon and claims the benefit of priority from Japanese patent application No. 2017-200218, filed on Oct. 16, 2017, the disclosure of which is incorporated herein in its entirety by reference.

### REFERENCE SIGNS LIST

100, 100A, 100B, 100C nurse operation assistance terminal
110 necessary item storage unit
120 speech output unit
130 receiving set (earphone, headphone)
140 microphone
150 transmitter
150A writing unit
150B terminal connector
160 speech recognition unit (speech identification means)
200, 200A, 200B, 200C recording device
210 nursing necessity degree table
220 receiver
220A reading unit
220B recording connector
230 speech recognition unit (speech identification means)
240 recording unit
300 recording medium
400, 400A, 400B, 400C nurse operation assistance terminal
410 microphone (collection unit)
420 transmitter (output means)
420A writing unit
420B terminal connector
430 treatment behavior acquisition device
432 speech recognition unit (speech identification means)
434 information extraction unit
500, 500A, 500B, 500C registration system
510 receiver
510A reading unit
510B registration connector
520 treatment behavior acquisition device
522 speech recognition unit (speech identification means)
524 information extraction unit
530 judgment device
532 information contrast unit
534 judgment unit
536 storage unit
538 learning unit
540 recording device
542 nursing necessity degree table
544 recording unit
600 recording medium
700 information processing apparatus
710 control unit (CPU)
720 storage unit
730 ROM
740 RAM
750 communication interface
760 user interface

1-5. (canceled)

6. A nurse operation assistance terminal comprising:
a collection unit configured to collect, as a collected speech, utterance generated by a nurse to a patient and/or conversation between the nurse and the patient in conjunction with a treatment behavior performed on the patient by the nurse at a treatment site; and
an output unit configured to output a collected speech signal indicative of the collected speech.

7. A nurse operation assistance system comprising:
the nurse operation assistance terminal described in claim 6; and
a registration system configured to receive the collected speech signal from the nurse operation assistance terminal,
wherein the registration system comprises:
a treatment behavior acquisition device configured to acquire treatment behavior information from the collected speech signal;
a judgment device configured to judge a nursing necessity degree based on the treatment behavior information to produce a judged value; and
a recording device configured to record the judged value.

8. The nurse operation assistance system as claimed in claim 7,
wherein the treatment behavior acquisition device comprises:
a speech identification unit configured to identify the collected speech signal to produce a speech identified result; and
an information extraction unit configured to extract the treatment behavior information from the speech identified result.

9. The nurse operation assistance system as claimed in claim 7,
wherein the judgment device comprises:
an information contrast means configured to contrast the treatment behavior information with predetermined necessary items to produce a plurality of keywords required to specify the nursing necessity degree; and
a judgment means configured to judge the nursing necessity degree, based on a judgment rule and using a combination of the plurality of keywords, to produce the judged value.

10. The nurse operation assistance system as claimed in claim 7,
wherein the judgment device comprises:
an information contrast unit configured to contrast the treatment behavior information with predetermined necessary items to produce a plurality of keywords required to specify the nursing necessity degree; and
a judgement unit configured to judge the nursing necessity degree based on a judgment rule using a combination of the plurality of keywords to produce the judged result.

11. The nurse operation assistance system as claimed in claim 10,
wherein the judgment device further comprises:
a learning unit configured to carry out machine-learning, responsive to the plurality of keywords and using, as teaching signals, data indicative of treatment behaviors actually performed in conjunction with a plurality of utterances generated by a plurality of nurses to a plurality of patients and/or a plurality of conversations exchanged between the plurality of nurses and the plurality of patients, to generate the judgment rule; and
a storage unit configured to store the generated judgment rule.

12. A nurse operation assistance method comprising:
acquiring treatment behavior information from a collected speech signal received from a nurse operation assistance terminal;
contrasting the treatment behavior information with predetermined necessary items to produce a plurality of keywords required to specify a nursing necessity degree;
judging the nursing necessity degree, based on a judgment rule and using a combination of the plurality of keywords, to produce a judged value; and
automatically recording the judged value in a corresponding predetermined field on a nursing necessity degree table.

13. The nurse operation assistance method as claimed in claim 12, further comprising:
carrying out machine-learning responsive to the plurality of keywords and using, as teaching signals, data indicative of treatment behaviors actually performed in conjunction with a plurality of utterances generated by a plurality of nurses to a plurality of patients and/or a plurality of conversations exchanged between the plurality of nurses and the plurality of patients, to generate the judgment rule; and
storing the generated judgment rule.

14. A non-transitory recording medium recording a nurse operation assistance program which causes a computer to execute:
a treatment behavior acquisition step of acquiring treatment behavior information from a collected speech signal received from a nurse operation assistance terminal;
an information contrast step of contrasting the treatment behavior information with predetermined necessary items to produce a plurality of keywords required to specify a nursing necessity degree;
a judgment step of judging the nursing necessity degree, based on a judgment rule and using a combination of the plurality of keywords, to produce a judged value; and
a recording step of automatically recording the judged value in a corresponding predetermined field on a nursing necessity degree table.

15. The non-transitory recording medium as claimed in claim 14, wherein the nurse operation assistance program causes the computer to further execute:
a learning step of carrying out machine-learning, responsive to the plurality of keywords and using, as teaching signals, data indicative of treatment behaviors actually performed in conjunction with a plurality of utterances generated by a plurality of nurses to a plurality of patients and/or a plurality of conversations exchanged between the plurality of nurses and the plurality of patients, to generate the judgment rule; and
a step of storing the generated judgment rule in a storage unit.

16-25. (canceled)
